(19) United States
(12) Patent Application Publication
(10) Pub. No.: US 2026/0124134 A1
(43) Pub. Date: May 7, 2026

(54) INJECTABLE LYOPHILIZED FORMULATION INCLUDING BETA-NICOTINAMIDE MONONUCLEOTIDE AND INJECTABLE FORMULATION INCLUDING THE SAME AND METHODS OF PREPARING THEM

(71) Applicant: Si Ha KANG, Seoul (KR)

(72) Inventor: Si Ha KANG, Seoul (KR)

(21) Appl. No.: 19/335,657

(22) Filed: Sep. 22, 2025

(30) Foreign Application Priority Data
Nov. 1, 2024 (KR) 10-2024-0153418
Jan. 31, 2025 (KR) 10-2025-0012349

(51) Int. Cl. A61K 9/00; A61K 9/08; A61K 9/19; A61K 31/706; A61K 47/26

(52) U.S. Cl. CPC A61K 9/0019; A61K 9/08; A61K 9/19; A61K 31/706; A61K 47/26

(57) ABSTRACT

The present invention relates to an injectable lyophilized formulation including beta-nicotinamide mononucleotide, an injectable formulation including the same, and methods of preparing them.

### CROSS-REFERENCE TO RELATED APPLICATION

This application claims priority to and the benefit of Korean Patent Application No. 10-2024-0153418, filed on Nov. 1, 2024 and Korean Patent Application No. 10-2025-0012349, filed on Jan. 31, 2025, the disclosure of which is incorporated herein by reference in its entirety.

### BACKGROUND

### 1. Field of the Invention

The present invention relates to an injectable lyophilized formulation including beta (β)-nicotinamide mononucleotide, an injectable formulation including the same, and methods of preparing them.

### 2. Discussion of Related Art

Globally, the increase in average life expectancy and improvements in living standards have intensified the demand for anti-aging or age-reversal. However, aging is the stage that follows maturity and is an inevitable process of all living organisms, occurring irreversibly over time and leading to death. Aging is influenced by genetic factors, lifestyle factors, and environmental factors. Although lifestyle and environmental improvements have occurred over the past centuries, the lack of a meaningful increase in maximum average life expectancy suggests that genetic factors paly the most critical role in aging. Taking this into consideration, improvement in genetic factors is expected to delay an aging rate and achieve the natural maximum lifespan. Among the genetic factors of aging, the accumulation of DNA damage is recognized, and maintenance of DNA stability is an important approach to suppressing aging and extending lifespan.

Nicotinamide mononucleotide (NMN) was identified as a potential rejuvenation drug in 2016 after the research team of Prof. Shinichiro Imai (Washington, United States) confirmed a 16% lifespan extension in mice orally administered NMN. NMN, existing in the body, activates sirtuins, longevity-related genes, and is reduced with aging. NMN is a nucleotide that is naturally generated through a reaction between a nucleoside containing ribose and nicotinamide (NAM) and a phosphate group. It has two isomeric forms, α-NMN and β-NMN, with the β isomer being the active form.

Such β-NMN has been marketed in the United States as an oral health supplement and nutraceutical. Since the oral formulation must pass through various digestive organs, only a part of the ingredient is absorbed, resulting in the need for a higher dosage. While sublingual administration exhibits the maximum 30% bioavailability, there is a problem of considerable interindividual variability depending on the characteristics of oral administration and the residence time under the tongue. In addition, the dosage of β-NMN nutraceuticals currently used in the United States is set at up to 1250 mg per day; however, due to the high cost of β-NMN, there is a limitation in consuming large amounts daily.

Compared to such oral administration, the administration of an injectable formulation is an optimal method to deliver an exact dose rapidly and in a well-controlled manner, thereby achieving systemic effects. In addition, such injectable formulations have the advantage of having the same effect with a smaller dose than oral administration. However, in this case, the stability and safety of injectable formulations should be given important consideration.

Therefore, in development of injectable formulations containing β-NMN, studies are needed to improve stability and safety.

### SUMMARY OF THE INVENTION

The present invention is directed to providing an injectable lyophilized formulation including β-NMN.

However, technical problems to be solved in the present invention are not limited to the above-described problems, and other problems which are not described herein will be fully understood by those of ordinary skill in the art from the following descriptions.

According to one aspect of the present invention, there is provided an injectable lyophilized formulation including β-NMN.

The injectable lyophilized formulation may further include one or more stabilizers selected from the group consisting of monosaccharides; disaccharides; and sugar alcohols.

The stabilizer may be i) one or more selected from the group consisting of mannitol, sorbitol, lactose, xylitol, trehalose, sucrose, maltose, and glucose, or ii) a combination of mannitol as a first stabilizer and one selected from the group consisting of xylitol, trehalose, sucrose, maltose, and glucose as a second stabilizer.

The injectable lyophilized formulation may have a pH of 3 to 8.

The water content of the injectable lyophilized formulation may be 5 (w/w) % or less.

In one embodiment of the present invention, an injectable formulation in which the lyophilized formulation is reconstituted in an infusion solution.

The injectable formulation may be for any one selected from the group consisting of intravenous, subcutaneous, intramuscular, intradermal, and intra-arterial administrations.

In the injectable formulation, the dose of the β-NMN may be administered in an amount of 1/20 to ½ of the amount determined to be therapeutically effective in oral administration.

In the injectable formulation, the β-NMN may be administered daily at a dose of 5 mg/kg to 40 mg/kg.

According to another aspect of the present invention, there is provided a method of preparing an injectable lyophilized formulation, which includes: (a) preparing a solution containing β-NMN; and (b) lyophilizing the solution.

According to still another aspect of the present invention, there is provided a method of preparing an injectable formulation, which includes: (a) preparing a solution containing β-NMN; (b) preparing an injectable lyophilized formulation by lyophilizing the solution; and (c) reconstituting the injectable lyophilized formulation by adding an infusion solution.

### BRIEF DESCRIPTION OF THE DRAWINGS

FIG. 1(A) shows the result of analyzing a blood NAD+ concentration in intravenous administration of a β-NMN injectable formulation prepared in Example 2 (IV formulation (2)), and FIG. 1(B) shows the result of analyzing a blood NADH concentration in intravenous administration of the β-NMN injectable formulation prepared in Example 2 (IV formulation (2)).

### DETAILED DESCRIPTION OF EXEMPLARY EMBODIMENTS

The present inventors confirmed that a lyophilized formulation (particularly, a lyophilized formulation maintaining the optimal pH condition) is employed to develop an injection containing β-NMN, thereby not only improving stability and safety but also achieving excellent animal pharmacokinetic (PK) evaluation results with a small dose, and thus completed the present invention.

Hereinafter, the present invention will be described in detail.

### Injectable Lyophilized Formulation/Injectable Formulation

The present invention provides an injectable lyophilized formulation containing β-NMN.

The present invention also provides an injectable formulation in which the lyophilized formulation is reconstituted in an infusion solution.

The injectable lyophilized formulation according to the present invention is a preliminary formulation for preparing an injectable formulation, which includes β-NMN.

Beta-nicotinamide mononucleotide is called β-NMN, and its specific structure is as follows:

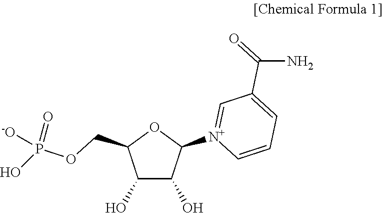

β-NMN is a NAD+ precursor, which may be converted into NAD+ in the body and has a variety of effects, including anti-aging, increased energy, improved cardiovascular health, protection of cognitive function, enhanced metabolism, and reduced inflammation and oxidative stress.

Particularly, according to the present invention, after the β-NMN is prepared in a lyophilized formulation, an injectable formulation is prepared. When the injectable formulation prepared as above is administered into the body, it may reduce NADH activity and increase NAD+ activity in the body, thereby maintaining the optimal ratio, leading to an excellent anti-aging related effect. That is, the injectable formulation improves the NAD+/NADH ratio in the body.

Meanwhile, when the β-NMN is prepared in a liquid formulation, not a lyophilized formulation, during the storage of the liquid formulation at 4° C. for 8 weeks, a change in the content of the β-NMN becomes relatively larger, leading to issues associated with decreased stability. In addition, when the injectable formulation is prepared from the liquid formulation, and particularly, when its pH is 6 or more, there is an issue of generating a significant amount of insoluble fine particles. Therefore, this injectable formulation has a limitation in that it is not effective in improving the NAD+/NADH ratio in the body.

The injectable lyophilized formulation may further include a stabilizer such that the hydrolysis of the β-NMN does not occur. As the stabilizer, those well known in the art may be employed, and include preferably one or more selected from the group consisting of monosaccharides (glucose, etc.); disaccharides (trehalose, sucrose, lactose, maltose, isomaltose, etc.); and sugar alcohols (mannitol, xylitol, sorbitol, maltitol, erythritol, lactitol, etc.), and more preferably sugar alcohols (mannitol, xylitol, sorbitol, maltitol, erythritol, lactitol, etc.), but the present invention is not limited thereto.

Specifically, the stabilizer may be i) one or more selected from the group consisting of mannitol, sorbitol, lactose, xylitol, trehalose, sucrose, maltose, and glucose, or ii) a combination of mannitol as a first stabilizer and one selected from the group consisting of xylitol, trehalose, sucrose, maltose, and glucose as a second stabilizer.

For example, the stabilizer may be mannitol alone as a first stabilizer. In addition, the stabilizer may include mannitol as the first stabilizer, in combination with xylitol, sorbitol, trehalose, sucrose, maltose, or glucose as the second stabilizer. Alternatively, the stabilizer may be xylitol, sorbitol, trehalose, sucrose, maltose, or glucose alone as the second stabilizer, instead of mannitol as the first stabilizer. Alternatively, the stabilizer may be the combination of xylitol and sorbitol, the combination of xylitol and sucrose, or the combination of sorbitol and maltose as the second stabilizer, instead of mannitol as the first stabilizer.

More specifically, the content of the stabilizer may be 10 wt % to 500 wt %, preferably 50 wt % to 200 wt %, and more preferably 90 wt % to 100 wt % with respect to the content of the β-NMN, but the present invention is not limited thereto.

The injectable lyophilized formulation may be maintained in an acidic condition, such as pH 3 to pH 8, preferably pH 3 or more and less than pH 6, and more preferably pH 3 to pH 5, but the present invention is not limited thereto. To satisfy the optimal pH condition, the injectable lyophilized formulation may further include hydrochloric acid (HCl); or optionally, one or more alkalizing agents selected from the group consisting of sodium hydroxide (NaOH), calcium hydroxide (Ca (OH) 2), potassium hydroxide (KOH), tromethamine, and sodium bicarbonate. For example, the injectable lyophilized formulation according to the present invention may employ the alkalizing agent in combination with hydrochloric acid (HCl), or may employ hydrochloric acid (HCl) alone without the alkalizing agent. Therefore, when the injectable lyophilized formulation is stored at 4° C. for 8 weeks, there is almost no change in the content of the β-NMN, leading to excellent stability. Meanwhile, when the injectable lyophilized formulation has a pH of 6 or more, during storage at 4° C. for 8 weeks, there is an issue of greatly increasing a change in the content of the β-NMN.

In addition, the injectable lyophilized formulation is in a state in which water content is minimized, which demonstrates that the water content may be 5 (w/w) % or less, preferably 3 (w/w) % or less, but the present invention is not limited thereto. The injectable lyophilized formulation may be formed in a cake with minimized water content.

In addition, the injectable lyophilized formulation according to the present invention must be reconstituted or diluted in an infusion solution to adjust the concentration of the β-NMN.

The infusion solution may be water for injection, or may further include an isotonic agent such as sodium chloride or glucose in water for injection (e.g., 0.1 to 2.0% physiological saline injection, or 1 to 10% glucose injection or lactose-added Ringer's solution).

Accordingly, the β-NMN in the injectable formulation according to the present invention may be maintained at a concentration of 1 mg/mL to 1,000 mg/mL, preferably 50 mg/mL to 100 mg/mL, but the present invention is not limited thereto. Therefore, the injectable formulation according to the present invention is suitable especially for intravenous administration

Meanwhile, the injectable formulation may be for administration selected from the group consisting of intravenous, subcutaneous, intramuscular, intradermal, and intra-arterial administrations, preferably intravenous administration, but the present invention is not limited thereto. Such intravenous administration can be rapidly administered at an exact dose and well controlled, thereby achieving systemic effects; however, for direct intravascular injection, stability and safety must be primarily considered.

In the injectable formulation, the dose of the β-NMN may be administered in an amount of 1/20 to ½, preferably 1/20 to ¼ of the amount determined to be therapeutically effective in oral administration. Despite administration at such a small dose, the β-NMN may exert an effect equal to or greater than that of the regular dose, and exhibit an immediate drug-release effect in the body.

In the injectable formulation, the β-NMN may be administered daily at a dose of 0.5 mg/kg to 500 mg/kg.

Blood samples are collected at 5, 30, 60, 120, and 360 minutes after administering the injectable formulation (after administering the β-NMN at 500 mg/kg/day for 3 days) and analyzed using a NAD+/NADH assay kit, revealing that i) the mean blood NAD+ concentrations may show an increase to 200% to 400% (particularly, 250% to 350%) at each time point compared to placebo administration, and ii) the mean blood NADH concentrations may show an increase to 150% to 300% (particularly, 150% to 250%) at each time point compared to placebo administration.

Particularly, blood samples are collected at 5, 30, and 60 minutes after administering the injectable formulation (after administering the β-NMN at 500 mg/kg/day for 3 days), and analyzed using a NAD+/NADH assay kit, revealing i) a 350% to 450% increase in the mean blood NAD+ concentration at each earlier time point, and ii) a 200% to 300% increase in the mean blood NADH concentration at each earlier time point, compared to placebo administration.

In addition, blood samples are collected at 30 minutes after administering the injectable formulation (after administering the β-NMN at 500 mg/kg/day for 3 days), and analyzed using a NAD+/NADH assay kit, revealing a 550% to 600% increase in the blood NAD+ concentration as compared to placebo administration. Meanwhile, blood samples are collected at 5 minutes after administering the injectable formulation, and then analyzed using a NAD+/NADH assay kit, revealing a 350% to 400% increase in the blood NADH concentration as compared to placebo administration.

That is, when the injectable formulation is administered, it can be seen that the increments in the mean and maximum blood NAD+ concentrations are comparatively larger than those of placebo administration. Particularly, the intravenous administration of the injectable formulation can optimize the NAD+/NADH ratio in the body, and thus can exhibit an excellent anti-aging related effect.

### Method of Preparing Injectable Lyophilized Formulation/Injectable Formulation

The present invention provides a method of preparing an injectable lyophilized formulation, which includes: (a) preparing a solution n containing β-NMN; and (b) lyophilizing the solution.

In addition, the present invention provides a method of preparing an injectable formulation, which includes: (a) preparing a solution containing β-NMN; (b) preparing an injectable lyophilized formulation by lyophilizing the solution; and (c) reconstituting the injectable lyophilized formulation by adding an infusion solution.

First, the present invention includes (a) preparing a solution containing β-NMN.

The solution includes β-NMN, which has been described above, and repetitive description will be omitted.

The solution may further include a stabilizer to prevent the hydrolysis of the β-NMN, and the stabilizer has also been described above, and therefore repetitive description will be omitted.

In this solution, nitrogen-substituted water for injection may be used as a solvent. The nitrogen-substituted water for injection may be prepared by a process known in the art, and has the benefit in cake formation following freeze-drying after oxygen is substituted with nitrogen through constitution and filling processes.

Next, the present invention includes (b) preparing an injectable lyophilized formulation by lyophilizing the solution.

Before lyophilization, stabilization may precede, which may be performed at 1° C. to 10° C. for 10 minutes to 200 minutes under atmospheric pressure.

The freeze-drying operation is divided into a freezing operation and a drying operation, wherein the freezing operation may be performed at −100° C. to −1° C. (preferably −80° C. to −5° C.) for 100 minutes to 1000 minutes under atmospheric pressure. To minimize bubble generation, the freezing operation is performed in two steps: The first step may be performed at −10° C. to −1° C.; and the second step may be performed at −100° C. to −50° C. Meanwhile, the drying operation may be performed at 5° C. to 50° C. (preferably 10° C. to 25° C.) for 500 minutes to 3000 minutes under 0.1 Pa to 1 Pa. To form a cake with minimized water content, the drying operation may be performed in two steps: The first step may be performed at 5° C. to 15° C.; and the second step may be performed at 20° C. to 50° C.

Since the injectable lyophilized formulation has been described above, repeated description will be omitted.

Next, the present invention includes (c) reconstituting the injectable lyophilized formulation by adding an infusion solution. The injectable formulation according to the present invention may thus be ultimately prepared.

The infusion solution is for adjusting the concentration of the β-NMN. Since the specific details have been provided earlier, repetitive description will be omitted.

Accordingly, the β-NMN in the injectable formulation according to the present invention may be maintained at a concentration of 50 mg/mL to 500 mg/mL. In addition, the injectable formulation according to the present invention may greatly reduce the number of insoluble fine particles even 24 hours after preparation, resulting in ensuring stability as an injection.

As seen from the above, the injectable lyophilized formulation according to the present invention (particularly a lyophilized formulation maintaining the optimal pH condition) includes β-NMN, and thus exhibits excellent stability.

Accordingly, when an injectable formulation is prepared by employing the injectable lyophilized formulation according to the present invention (particularly a lyophilized formulation maintaining the optimal pH condition), its stability and safety can be improved. Furthermore, compared to oral administration, it has the advantage of exhibiting excellent efficacy even with a smaller dose.

In addition, the injectable formulation according to the present invention may provide an optimized NAD+/NADH ratio in the body, and may exhibit an excellent anti-aging related effect.

Hereinafter, preferred examples will be presented to allow the present invention to be better understood. However, the following examples are merely provided in order for the present invention to be more easily understood, and the content of the present invention is not limited by the following examples.

### EXAMPLES

### Examples 1 to 3: Preparation of Injectable Lyophilized Formulations for β-NMN Injection

67 g of β-NMN and 60 g of mannitol were added to 80 mL of water for injection and stirred for 60 minutes to constitute, the pH was adjusted, and then 100 mL of the solution was collected. The collected solution was filtered using a 0.2 μm cellulose acetate (CA) filter. 5 mL of the filtered solution was put into a 20 mL vial, and lyophilized using a freeze-drying apparatus (JABA Lyoph-Pride LP20), thereby preparing a β-NMN injectable lyophilized formulation (water content≤5 (w/w) %) as shown in Table 1. Here, lyophilization was performed by 1) a loading process at 5° C. under atmospheric pressure; 2) a stabilization process at 5° C. for 120 minutes under atmospheric pressure; 3) a freezing process at −25° C. for 720 minutes under atmospheric pressure; 4) a primary drying process at 10° C. for 1440 minutes under 0.2 Pa; and 5) a secondary drying process at 25° C. for 500 minutes under 0.2 Pa.

### Comparative Examples 1 to 3: Preparation of Liquid Formulation for β-NMN Injection

A liquid formulation for β-NMN injection was prepared as in Table 1 in the same manner as in Examples 1 to 3, except that lyophilization was omitted.

TABLE 1

| | | | | Liquid formulation | | |
|---|---|---|---|---|---|---|
| | Lyophilized formulation | | | Comparative | Comparative | Comparative |
| Classification | Example 1 | Example 2 | Example 3 | Example 1 | Example 2 | Example 3 |
| β-NMN | 67 g | 67 g | 67 g | 67 g | 67 g | 67 g |
| Mannitol | 60 g | 60 g | 60 g | 60 g | 60 g | 60 g |
| HCl | q.s. | q.s. | q.s. | q.s. | q.s. | q.s. |
| Sodium | — | q.s. | q.s. | q.s. | q.s. | q.s. |
| hydroxide | | | | | | |
| pH | pH 3 to 4 | pH 4 to 5 | pH 6 to 7 | pH 3 to 4 | pH 4 to 5 | pH 6 to 7 |

### Experimental Example 1: Evaluation of Stability of Each Formulation for β-NMN Injection

To evaluate the stability of the lyophilized formulations prepared in Examples 1 to 3 and the liquid formulations prepared in Comparative Examples 1 to 3, changes in β-NMN content were measured at 4° C. for 8 weeks as below. The results are shown in FIG. 2.
Equipment: LC-PDA (Waters HPLC system)
Conditions: column (GL Sciences InerSustain C18, 250×4.6 mm, 5 μm), mobile phase (10 mM phosphate-buffered saline (pH 3.0)/methanol=90/10), flow rate (1.0 mL/min), injection amount (20 μL)

TABLE 2

| | | | | | Liquid formulation | | |
|---|---|---|---|---|---|---|---|
| | | Lyophilized formulation | | | Comparative | Comparative | Comparative |
| Classification | | Example 1 | Example 2 | Example 3 | Example 1 | Example 2 | Example 3 |
| Change | Initial | 99.8% | 99.1% | 94.2% | 100.1% | 99.3% | 95.2% |
| in content | 4 weeks | 99.7% | 99.5% | 90.3% | 95.3% | 93.3% | 80.2% |
| of β-NMN | 8 weeks | 99.9% | 99.2% | 85.3% | 90.2% | 83.3% | 40.8% |

As shown in Table 2, it can be seen that, compared to the liquid formulations prepared in Comparative Examples 1 to 3, the lyophilized formulations prepared in Examples 1 to 3 exhibit excellent stability although they show negligible changes in β-NMN content at 4° C. for 8 weeks. Particularly, when the lyophilized formulations prepared in Examples 1 to 3 are maintained at pH 3 to 5, changes in β-NMN content at 4° C. for 8 weeks are within 1%, indicating particularly excellent stability.

### Experimental Example 2: Evaluation of Stability of Formulations for β-NMN Injection

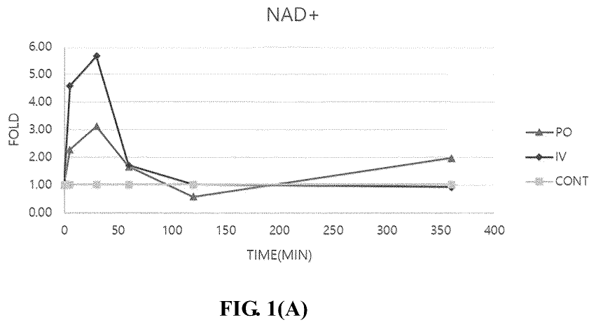

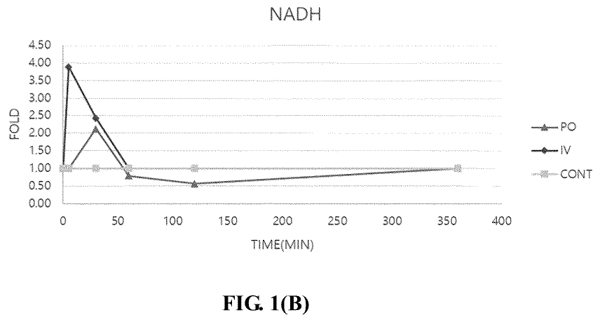

A β-NMN injectable formulation containing 67 mg/ml of β-NMN was prepared by adding water for injection to each of the lyophilized formulations prepared in Examples 1 to 3 and the liquid formulations prepared in Comparative Examples 1 to 3. After 24 hours, the appearance and insoluble fine particles (impurities of 10 μm, 25 μm, 50 μm or larger) were observed at 25° C. to evaluate stability as an injection. Here, the appearance was visually observed, and the number of insoluble fine particles was measured by a microscopic counting method. The results are shown in Table 3 and FIG. 1.

TABLE 3

| | | | | | Liquid formulation | | |
|---|---|---|---|---|---|---|---|
| | | Lyophilized formulation | | | Comparative | Comparative | Comparative |
| Classification | | Example 1 | Example 2 | Example 3 | Example 1 | Example 2 | Example 3 |
| Appearance | | Clear | Clear | Light brown | Clear | Clear | Light brown |
| | | solution | solution | solution | solution | solution | solution |
| Insoluble | 10 μm | 0 | 0 | 43 | 0 | 0 | 76 |
| fine | 25 μm | 0 | 0 | 2 | 0 | 0 | 4 |
| particles | 50 μm | 0 | 0 | 0 | 0 | 0 | 0 |

As shown in Table 3, it can be seen that, compared to injectable formulations in which the liquid formulations prepared in Comparative Examples 1 to 3 were reconstituted, injectable formulations in which the lyophilized formulations prepared in Examples 1 to 3 were constituted were able to significantly reduce the number of insoluble fine particles 24 hours after reconstitution, resulting in achieved stability and safety as an injection. Particularly, when any of the lyophilized formulations prepared in Examples 1 to 3 is maintained at pH 3 to 5, the injectable formulation in which it was reconstituted is observed in a clear solution state, confirming that no insoluble fine particles are generated. Accordingly, it can be seen that they have excellent stability and safety as an injection.

### Experimental Example 3: Evaluation of Stability of Lyophilized Formulations/Injectable Formulations Depending on Different Stabilizer for Formulation for β-NMN Injection

### (1) Evaluation of Stability of Lyophilized Formulations/Injectable Formulations by Adjusting “Mannitol” Content

TABLE 4

| | | Lyophilized formulation | |
|---|---|---|---|
| Classification | | Example 4 | Example 5 |
| β-NMN | | 600 mg | 600 mg |
| Stabilizer | | Mannitol 300 mg | Mannitol 1,200 mg |
| HCl | | q.s. | q.s. |
| Sodium hydroxide | | q.s. | q.s. |
| pH | | pH 3 to 5 | pH 3 to 5 |
| Evaluation | Change in β- | 0.8 | 0.9 |
| of stability | NMN | | |
| of | content (%) | | |
| lyophilized | at 4° C. | | |
| formulation | for 8 weeks | | |
| Evaluation | Appearance | Clear solution | Clear solution |
| of stability | right after | | |
| of | reconstitution | | |
| injectable | Appearance | Clear solution | Clear solution |
| formulation | at 25° C. | | |
| | for 8 weeks | | |
| | Insoluble | 2 | 2 |
| | fine particles | | |
| | (10 μm) at | | |
| | 25° C. for 8 | | |
| | weeks | | |
| | (25 μm) | 0 | 0 |
| | (50 μm) | 0 | 0 |

As shown in Table 4, the lyophilized formulations according to Examples 4 and 5 are examples in which mannitol was employed as a stabilizer like Examples 1 and 2 in the specification of the original application, and its content was adjusted. In this case, at 4° C. for 8 weeks, the change in β-NMN content is maintained within 0.9%, confirming that substantially excellent stability is achieved.

In addition, injectable formulations in which the lyophilized formulation was reconstituted according to Examples 4 and 5 remain as a clear solution at 25° C. for up to 8 weeks, and generate traces of insoluble fine particles, considering that that they have excellent stability and safety as an injection.

### (2) Evaluation of Stability of Lyophilized Formulations/Injectable Formulations by Employing Combination of “Mannitol” as First Stabilizer and Second Stabilizer

TABLE 5

| | | Lyophilized formulation | | | | | |
|---|---|---|---|---|---|---|---|
| Classification | | Example 6 | Example 7 | Example 8 | Example 9 | Example 10 | Example 11 |
| β-NMN | | 600 mg | 600 mg | 600 mg | 600 mg | 600 mg | 600 mg |
| Stabilizer | | Mannitol | Mannitol | Mannitol | Mannitol | Mannitol | Mannitol |
| | | 150 mg, | 150 mg, | 150 mg, | 150 mg, | 150 mg, | 150 mg, |
| | | Xylitol | Sorbitol | Trehalose | Sucrose | Maltose | Glucose |
| | | 150 mg | 150 mg | 150 mg | 150 mg | 150 mg | 150 mg |
| HCl | | q.s. | q.s. | q.s. | q.s. | q.s. | q.s. |
| Sodium hydroxide | | q.s. | q.s. | q.s. | q.s | q.s. | q.s. |
| pH | | pH 3 to 5 | pH 3 to 5 | pH 3 to 5 | pH 3 to 5 | pH 3 to 5 | pH 3 to 5 |
| Evaluation | Change in β- | 0.9 | 0.9 | 1.0 | 1.1 | 1.2 | 1.2 |
| of stability | NMN | | | | | | |
| of | content (%) | | | | | | |
| lyophilized | at 4° C. for 8 | | | | | | |
| formulation | weeks | | | | | | |
| Evaluation | Appearance | Clear | Clear | Clear | Clear | Clear | Clear |
| of stability | right after | solution | solution | solution | solution | solution | solution |
| of | reconstitution | | | | | | |
| injectable | Appearance | Clear | Clear | Clear | Clear | Clear | Clear |
| formulation | at 25° C. for 8 | solution | solution | solution | solution | solution | solution |
| | weeks | | | | | | |
| | Insoluble | 3 | 3 | 8 | 9 | 9 | 12 |
| | fine particles | | | | | | |
| | (10 μm) at | | | | | | |
| | 25° C. for 8 | | | | | | |
| | weeks | | | | | | |
| | (25 μm) | 0 | 0 | 0 | 0 | 0 | 0 |
| | (50 μm) | 0 | 0 | 0 | 0 | 0 | 0 |

As shown in Table 5, the lyophilized formulations according to Examples 6 to 11 are examples that employed mannitol as a first stabilizer in combination with xylitol, sorbitol, trehalose, sucrose, maltose, or glucose as a second stabilizer.

That is, the lyophilized formulations according to Examples 6 to 11 maintain changes in β-NMN content within 1.2% at 4° C. for 8 hours even by employing mannitol as a first stabilizer, in combination with a second stabilizer (xylitol, sorbitol, trehalose, sucrose, maltose, or glucose), confirming that they have excellent stability.

In addition, the injectable formulations in which the lyophilized formulations according to Examples 6 to 11 were reconstituted remain a clear solution at 25° C. for up to 8 weeks, and generate traces of insoluble fine particles, considering that they have excellent stability and safety as an injection.

### (3) Evaluation of Stability of Lyophilized Formulations/Injectable Formulations by Employing Second Stabilizer Alone, Instead of “Mannitol” as First Stabilizer

TABLE 6

| | | Lyophilized formulation | | | | | |
|---|---|---|---|---|---|---|---|
| Classification | | Example 12 | Example 13 | Example 14 | Example 15 | Example 16 | Example 17 |
| β-NMN | | 600 mg | 600 mg | 600 mg | 600 mg | 600 mg | 600 mg |
| Stabilizer | | Xylitol | Sorbitol | Trehalose | Sucrose300 | Maltose | Glucose |
| | | 300 mg | 300 mg | 300 mg | mg | 300 mg | 300 mg |
| HCl | | q.s. | q.s. | q.s. | q.s. | q.s. | q.s. |
| Sodium hydroxide | | q.s. | q.s. | q.s. | q.s. | q.s. | q.s. |
| pH | | pH 3 to 5 | pH 3 to 5 | pH 3 to 5 | pH 3 to 5 | pH 3 to 5 | pH 3 to 5 |
| Evaluation | Change in β- | 1.0 | 1.2 | 1.2 | 1.3 | 1.4 | 1.5 |
| of stability | NMN | | | | | | |
| of | content (%) | | | | | | |
| lyophilized | at 4° C. for 8 | | | | | | |
| formulation | weeks | | | | | | |
| Evaluation | Appearance | Clear | Clear | Clear | Clear | Clear | Clear |
| of stability | right after | solution | solution | solution | solution | solution | solution |
| of | reconstitution | | | | | | |
| injectable | Appearance | Clear | Clear | Clear | Clear | Clear | Clear |
| formulation | at 25° C. for 8 | solution | solution | solution | solution | solution | solution |
| | weeks | | | | | | |
| | Insoluble | 10 | 12 | 15 | 18 | 20 | 30 |
| | fine particles | | | | | | |
| | (10 μm) at | | | | | | |
| | 25° C. for 8 | | | | | | |
| | weeks | | | | | | |
| | (25 μm) | 0 | 0 | 0 | 0 | 0 | 0 |
| | (50 μm) | 0 | 0 | 0 | 0 | 0 | 0 |

As shown in Table 6, the lyophilized formulations according to Examples 12 to 17 are examples in which xylitol, sorbitol, trehalose, sucrose, maltose, or glucose alone was employed as a second stabilizer, instead of mannitol as a first stabilizer. That is, the lyophilized formulations according to Examples 12 to 17 in which a second stabilizer is employed alone (xylitol, sorbitol, trehalose, sucrose, maltose, or glucose), instead of mannitol as a first stabilizer, maintain changes in β-NMN content within 1.5% at 4° C. for 8 weeks, confirming that they have excellent stability.

In addition, injectable formulations in which the lyophilized formulations according to Examples 12 to 17 were reconstituted remain as a clear solution at 25° C. for up to 8 weeks, and generate traces of insoluble fine particles, considering that that they have excellent stability and safety as an injection.

### (4) Evaluation of Stability of Lyophilized Formulations/Injectable Formulations by Employing Combination of Second Stabilizers, Instead of “Mannitol” as First Stabilizer

TABLE 7

| | | Lyophilized formulation | | |
|---|---|---|---|---|
| Classification | | Example 18 | Example 19 | Example 20 |
| β-NMN | | 600 mg | 600 mg | 600 mg |
| Stabilizer | | Xylitol 150 mg, | Xylitol 150 mg, | Sorbitol 150 mg, |
| | | Sorbitol 150 mg | Sucrose 150 mg | Maltose 150 mg |
| HCl | | q.s. | q.s. | q.s. |
| Sodium hydroxide | | q.s. | q.s. | q.s. |
| pH | | pH 3 to 5 | pH 3 to 5 | pH 3 to 5 |
| Evaluation | Change in β- | 1.1 | 1.2 | 1.3 |
| of stability | NMN | | | |
| of | content (%) | | | |
| lyophilized | at 4° C. | | | |
| formulation | for 8 weeks | | | |
| Evaluation | Appearance | Clear solution | Clear solution | Clear solution |
| of stability | right after | | | |
| of | reconstitution | | | |
| injectable | Appearance | Clear solution | Clear solution | Clear solution |
| formulation | at 25° C. | | | |
| | for 8 weeks | | | |
| | Insoluble | 10 | 15 | 18 |
| | fine particles | | | |
| | (10 μm) at | | | |
| | 25° C. for 8 | | | |
| | weeks | | | |
| | (25 μm) | 0 | 0 | 0 |
| | (50 μm) | 0 | 0 | 0 |

As shown in Table 7, the lyophilized formulations according to Examples 18 to 20 are examples in which the combination of xylitol and sorbitol, the combination of xylitol and sucrose, or the combination of sorbitol and maltose was employed as the second stabilizer, instead of mannitol as the first stabilizer. That is, for the lyophilized formulations according to Examples 18 to 20 in which the combination of the second stabilizers (the combination of xylitol and sorbitol, the combination of xylitol and sucrose, and the combination of sorbitol and maltose) was employed, instead of mannitol as the first stabilizer, changes in β-NMN content were maintained within 1.3% at 4° C. for 8 weeks, confirming that they have excellent stability.

In addition, the injectable formulations in which the lyophilized formulations according to Example 18 to 20 were reconstituted remain a clear solution at 25° C. for 8 weeks and generate traces of insoluble fine particles, considering that they have excellent stability and safety as an injection.

### Experimental Example 4: Animal Pharmacokinetic (PK) Evaluation of Formulation for β-NMN Injection (IV Formulation (1)

A β-NMN injectable formulation in which the β-NMN concentration was 67 mg/mL was prepared by adding water for injection to the lyophilized formulation prepared in Example 2 (pH 4 to 5). The prepared β-NMN injectable formulation was intravenously administered to rats at 40 mg/kg, 20 mg/kg, and 10 mg/kg to assess blood NAD levels for 6 hours. Meanwhile, the lyophilized formulation prepared in Example 2 (pH 4 to 5) was prepared as an oral formulation and orally administered to rats at 80 mg/kg, and 40 mg/kg to assess blood NAD levels for 6 hours. Blood samples (0.5 mL each) were collected before administration, and 5, 10, 15, 30, 45, 60, and 90 minutes and 2, 4, and 6 hours after administration, and blood NAD levels were assessed as below. The result is shown in Table 8.
Equipment: LC-MS/MS (Nexera X2 Ultra HPLC system), triple quadrupole mass spectrometer (LCMS-8060, Shimadzu)
Conditions: column (NMN-2 150 mm 2.0 mm, 2.2 μm, Shimadzu), 4° C., mobile phase (acetonitrile (A), 0.1% formic acid (B)), mobile phase conditions (1% B from 0 to 2 minutes, 1 to 38.6 B from 2 to 10 minutes, 95% B from 10 to 12 minutes, and 1% B from 12 to 15 minutes), flow rate (0.2 mL/min), injection amount (2 μL)
Analyzer conditions: single reaction monitoring, 0.4 seconds (1 ms pause), retention time (10-15 ms), interface voltage (3.5 KV), nebulizing gas flow (3 L/min), heating gas (10 L/min), drying gas flow (10 L/min), interface temperature (300° C.), desolvation line temperature (200° C.), heating block temperature (350° C.), collision-induced dissociation gas pressure (270 Kpa)

TABLE 8

| Classification | Lyophilized formulation | | | Oral formulation | |
|---|---|---|---|---|---|
| Dose (mg/kg) | 40 | 20 | 10 | 160 | 80 |
| AUC (ng/mL*hr) | 4589 | 3254 | 1495 | 3254 | 1326 |
| Cmax (min) | 5 | 5 | 5 | 90 | 90 |
| T1/2 (min) | 24 | 24 | 24 | 30 | 30 |

As shown in Table 8, it is confirmed that an injectable formulation in which the lyophilized formulation prepared in Example 2 was reconstituted exhibits an equivalent or superior effect even when administered at ½ or less (particularly ⅛ or less) compared to oral formulation. Particularly, in the case of intravenous administration, due to the immediate drug release effect in the body, it can have a faster effect compared to oral administration.

### Experimental Example 5: Analysis of Blood NAD+/NADH Concentrations in Intravenous Administration of Formulation for β-NMN Injection (IV Formulation (2))

A β-NMN injectable formulation in which the β-NMN concentration is 67 mg/mL was prepared by adding water for injection to the lyophilized formulation (pH 4 to 5) prepared in Example 2.

At 5, 30, 60, 120, and 360 minutes after the formulation was intravenously administered to rats at a β-NMN dose of 500 mg/kg/day for 3 days (IV formulation (2)), blood samples were collected, and blood NAD+ and NADH concentrations were measured at each time point using an ABCAM NAD+/NADH assay kit (Cat No. ab65348).

Here, these concentrations were compared with those of the oral administration of the β-NMN-only-containing formulation to a rat at the same dose (500 mg/kg/day) for 3 days (PO formulation), and an β-NMN-free injectable formulation as a placebo intravenously administered to a rat for 3 days was prepared as a control. The results are shown in Tables 9 and 10, and FIGS. 1(A) and 1(B).

TABLE 9

| | Blood NAD+ concentration | | | | | | | |
|---|---|---|---|---|---|---|---|---|
| | 0 | 5 | 30 | 60 | 120 | 360 | | Initial |
| Classification | min | min | min | min | min | min | Mean | mean |
| IV | 1.00 | 4.58 | 5.66 | 1.69 | 1.00 | 0.91 | 2.77 | 4.00 |
| PO | 1.00 | 2.27 | 3.12 | 1.65 | 0.56 | 1.95 | 1.91 | 2.35 |
| CONT | 1.00 | 1.00 | 1.00 | 1.00 | 1.00 | 1.00 | 1.00 | 1.00 |

As shown in Table 9 and FIG. 1(A), for the IV formulation (2), the mean blood NAD+ concentration shows an increase to 200% to 400% (particularly 250% to 350%) at each time point, which is a significantly higher value than that of the PO formulation. Particularly, it is confirmed that, at the earlier time points (5, 30, and 60 minutes), the mean blood NAD+ concentrations are significantly increased to 350% to 450%, compared to those of the placebo administration. In addition, the IV formulation (2) shows the maximum blood NAD+ concentration at 30 minutes, and this value was confirmed to be increased to 566% compared to that of the placebo administration.

TABLE 10

| | Blood NADH concentration | | | | | | | |
|---|---|---|---|---|---|---|---|---|
| | 0 | 5 | 30 | 60 | 120 | 360 | | Initial |
| Classification | min | min | min | min | min | min | Mean | mean |
| IV | 1.00 | 3.88 | 2.43 | 1.00 | 1.00 | 1.00 | 1.86 | 2.43 |
| PO | 1.00 | 1.00 | 2.12 | 0.78 | 0.56 | 1.00 | 1.09 | 1.30 |
| CONT | 1.00 | 1.00 | 1.00 | 1.00 | 1.00 | 1.00 | 1.00 | 1.00 |

As shown in Table 10 and FIG. 1(B), it is confirmed that the IV formulation (2) shows the mean blood NADH concentration relatively increased to 150% to 300% (particularly 150% to 250%) at each time point, compared to those of the placebo administration. This value is considered to be a significantly higher value than that of the PO formulation. Particularly, at the earlier time points (5, 30, and 60 minutes), the mean blood NADH concentrations are confirmed to be increased to 200% to 300%, compared to those of the placebo administration. In addition, it is confirmed that the IV formulation (2) shows the maximum blood NADH concentration at 5 minutes, which is increased to 388% compared to that of the placebo administration.

Summarizing the above results, compared to the placebo administration, the IV formulation may show that the increments in the mean and maximum blood NAD+ concentrations are relatively higher than the increments in the mean and maximum blood NADH concentrations. That is, as the injectable formulation using the β-NMN lyophilized formulation is implemented as the IV formulation, the NAD+/NADH ratio may be optimized in the body, leading to an excellent anti-aging related effect.

An injectable lyophilized formulation according to the present invention (particularly a lyophilized formulation maintaining the optimal pH condition) includes β-NMN, and thus exhibits excellent stability.

Accordingly, when an injectable formulation is prepared by employing the injectable lyophilized formulation according to the present invention (particularly the lyophilized formulation maintaining the optimal pH condition), it may have an advantage of improved stability and safety. Moreover, it has the advantage of exhibiting excellent efficacy even with a smaller dose than oral administration.

In addition, the injectable formulation according to the present invention can optimize a NAD+/NADH ratio in the body, thereby exhibiting an excellent anti-aging related effect.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative in all aspects and not restrictive.

What is claimed is:

1. An injectable lyophilized formulation comprising beta-nicotinamide mononucleotide.

2. The injectable lyophilized formulation of claim 1, further comprising:
one or more stabilizers selected from the group consisting of monosaccharides; disaccharides; and sugar alcohols.

3. The injectable lyophilized formulation of claim 2, wherein the stabilizer is i) one or more selected from the group consisting of mannitol, sorbitol, lactose, xylitol, trehalose, sucrose, maltose, and glucose, or
ii) a combination of mannitol as a first stabilizer and one selected from the group consisting of xylitol, trehalose, sucrose, maltose, and glucose as a second stabilizer.

4. The injectable lyophilized formulation of claim 1, which has a pH of 3 to 8.

5. The injectable lyophilized formulation of claim 1, wherein the water content of the injectable lyophilized formulation is 5 (w/w) % or less.

6. An injectable formulation in which the injectable lyophilized formulation of claim 1 is reconstituted in an infusion solution.

7. The injectable formulation of claim 6, which is for any one selected from the group consisting of intravenous, subcutaneous, intramuscular, intradermal, and intra-arterial administrations.

8. The injectable formulation of claim 6, wherein the dose of the beta-nicotinamide mononucleotide is administered in an amount of 1/20 to ½ of the amount determined to be therapeutically effective in oral administration.

9. The injectable formulation of claim 6, wherein the beta-nicotinamide mononucleotide is administered daily at a dose of 0.5 mg/kg to 500 mg/kg.

10. The injectable formulation of claim 6, wherein blood samples are collected at 5, 30, 60, 120, and 360 minutes after the administration of the injectable formulation to analyze using a NAD+/NADH assay kit, revealing that
i) the mean blood NAD+ concentrations show an increase to 200% to 400% at each time point compared to placebo administration, and
ii) the mean blood NADH concentrations show an increase to 150% to 300% at each time point, compared to placebo administration.

11. A method of preparing an injectable lyophilized formulation, comprising:
(a) preparing a solution containing beta-nicotinamide mononucleotide; and
(b) lyophilizing the solution.

12. A method of preparing an injectable formulation, comprising:
(a) preparing a solution containing beta-nicotinamide mononucleotide;
(b) preparing an injectable lyophilized formulation by lyophilizing the solution; and
(c) reconstituting the injectable lyophilized formulation by adding an infusion solution.
